(19) United States
(12) Patent Application Publication
(10) Pub. No.: US 2014/0224249 A1
(43) Pub. Date: Aug. 14, 2014

(54) Apparatus for Administering Volatilizable Medicaments

(71) Applicant: Forrest Vaughan Landry, Del Mar, CA (US)

(72) Inventor: Forrest Vaughan Landry, Del Mar, CA (US)

(21) Appl. No.: 13/766,027

(22) Filed: Feb. 13, 2013

(51) Int. Cl. A61M 16/14; A61M 15/06; A61M 16/10

(52) U.S. Cl. CPC A61M 16/145; A61M 16/109; A61M 15/06
USPC 128/203.26

(57) ABSTRACT

An apparatus is disclosed for administering a volatilizable medicament, comprising a thermally stable, chemically inert, highly porous matrix media, preferably fused or sintered quartz wool, housed in a small container, which can be heated to high temperatures to volatilize the active ingredients for inhalation by a user.

### BACKGROUND OF THE INVENTION

The present invention relates generally to the field of apparatuses and methods for administering medicaments having volatilizable active ingredients, by volatilizing the active ingredients and introducing them into an air stream, for inhalation by a person.

Previous approaches for administering volatilizable medicaments have generally involved putting a liquid, resin or paste with the active ingredients into a small pan or bowl. The pan or bowl is heated using a flame or other heat source, to vaporize at least some of the active ingredients. The user then inhales the vapors containing the active ingredients.

However, this general approach has some important drawbacks and disadvantages. First, because only a portion of the mass of liquid or resin is in direct contact with the heating surface of the pan or bowl, different portions of the mass of medicament will be at different temperatures. This can lead to uneven heat and temperature distributions throughout the mass, and to uneven vaporization from the mass. Also, with a fairly viscous medicament, the mass can harden due to overcooking in some portions of the mass, before all of the active ingredients have been vaporized from the overall mass.

Also, this approach requires the user to measure and apply the correct dosage amount to the pan or bowl, which can lead to errors in administration of a specific intended dose.

Also, because the bowl or tray is generally open at the top, excessive tipping of the bowl or tray can cause spillage of medicament and loss of at least a portion of the dose.

Finally, it is desirable to have a device that will heat and cool rapidly, so it will reach vaporization temperatures quickly and efficiently and will then rapidly cool down to a safe temperature. However, because these prior art devices tend to be fairly large and thick and substantial in terms of their physical structures, they tend to heat slowly and cool slowly.

Electronic cigarettes have also been developed, which operate on the principle of vaporizing a liquid nicotine solution to create a vaporized nicotine stream, which the user can inhale. Electronic cigarettes share three essential components: (1) a “cartridge” that serves as a mouthpiece and usually doubles as a small reservoir holding the liquid that is to be vaporized; (2) an “atomizer” that serves as the heating element responsible for vaporizing the liquid; and (3) a power supply, which in portable models is a battery. Other electronic components necessary for operation are housed within the power unit.

A “cartomizer” option is available for most models of electronic cigarettes, which replaces the separate cartridge and atomizer components with a single integrated piece. This option is disposable, as opposed to standalone atomizers, which are reusable and comparatively expensive. The cartridge is a small, usually disposable plastic container, with openings on each end. One end is placed in the user's mouth, while the other attaches to the atomizer (heating element). This component serves as both a liquid reservoir and mouthpiece, and as such, must allow the passage of liquid to the atomizer, as well as vapor from the atomizer back to the user's mouth, without allowing liquid into the mouth. This is usually accomplished via an absorbent sponge-like material to keep the liquid in place, resting on a plastic barrier separating it from the mouth-end opening. The mouthpiece casing is constructed with side channels that allow vapor to pass from the atomizer, around the liquid chamber, to the mouth-end opening. When the liquid in a cartridge has been depleted, the user can usually choose between refilling it, or replacing it with another pre-filled cartridge.

The atomizer of an electronic cigarette is the heating element responsible for vaporizing the liquid, and generally consists of a simple filament and a metal mesh wick that draws the liquid into the atomizer. The atomizer is positioned in the center of the three components that make up the entire electronic cigarette cylinder, as the cartridge attaches to one end, and the power unit to the other.

Most portable electronic cigarette power units contain a lithium-ion rechargeable battery. The housing for the power source and electronic circuitry is usually the largest component of an electronic cigarette.

Some electronic cigarettes contain an electronic airflow sensor, in the case of “automatic” electronic cigarettes, so that activation is triggered simply by drawing breath through the device. Other “manual” electronic cigarette power units are constructed with a button that activates the heating element, and must be held during operation. A timed cutoff switch to prevent overheating, and/or a colored LED to announce activation, may also be included in the power unit casing.

Electronic cigarettes do, however, suffer from some disadvantages and drawbacks. First, the atomizer's filament tends to lose efficiency over time due to a buildup of sediment, or “burns out” entirely, requiring replacement. This creates one of the primary recurring expenses associated with electronic cigarettes.

Second, the liquid nicotine is stored separately, in a different location from the atomizer in which it is vaporized. This requires a separate liquid nicotine container as a main component of the device, which can be difficult to refill.

Under the circumstances, and in view of the above-described deficiencies in previously known methods for administering dosages of medicaments with volatilizable active ingredients, there is a need for an apparatus that will provide a simple and efficient way to administer such medicaments.

There also is a need for an apparatus that is simple to use and that will allow the efficient, clean vaporization of the active ingredient(s) in a medicament that is to be administered to a person.

There also is a need for an apparatus that is simple to use, and that can be used in combination with a variety of common types of herbal vaporizers, water pipes or standard pipes, through which the vapors with the active ingredients can be inhaled by the user as part of the vaporizer's or water pipe's or pipe's normal air stream.

There also is a need for an apparatus that can be pre-loaded with a dosage unit of an active ingredient, before the dosage unit is provided to a human end-user.

There also is a need for an apparatus that can be reused and re-loaded with another dose of an active ingredient, after the apparatus has been used to administer a previous dose.

There also is a need for an apparatus that is made of chemically inert materials and materials that will not be thermally degraded or affected by exposure to high temperatures as described herein.

Finally, there also is a need for an apparatus that can be heated to high temperatures, usually in excess of 650 F, to clean the entire device and to drive away any remaining medicament after use, so the device can be recharged and reused in its original form.

### BRIEF SUMMARY OF THE INVENTION

These and a number of additional objects are met by the present invention. The present invention relates generally to the field of apparatuses for administering medicaments that have volatilizable active ingredients, by volatilizing the active ingredients, so they can be inhaled by a person.

The medicament is applied to a thermally stable, chemically inert, highly porous matrix media having channels or interstitial spaces within the matrix material. As used herein, the term “thermally stable” means that a material is inflammable and non-combustible throughout the operational range of temperatures discussed herein, and is not physically or chemically altered or degraded by exposure to such temperatures. The term “chemically inert” means the material will not chemically react with the types of chemicals one would expect to be present in using the apparatus, including any solvents or substances that might be used to carry the active ingredients.

The medicament is applied to the matrix material in liquid, resin, wax or paste form, and permeates into the channels or interstitial spaces within the matrix material. If the medicament is in the form of a paste, wax, resin or some other non-liquid form, the permeation can be aided by heating the matrix and the medicament to decrease the viscosity of the paste, wax or resin.

The matrix material can then be heated to high temperatures to volatilize the active ingredients, by placing the matrix material and any small container for it into the heating chamber of a vaporizer, water pipe or pipe, and using that device to heat the matrix material. The volatile active ingredients are entrained into the device's normal air stream, and can be inhaled by a person using the device.

### DESCRIPTION OF THE SEVERAL VIEWS OF THE DRAWING

FIG. 1 of the drawing shows one possible embodiment of a matrix material (made of fibrous quartz wool) for use in dispensing medicaments.

FIG. 2 of the drawing shows a perspective overall view of the container portion of an apparatus made in accordance with the present invention.

FIG. 3 of the drawing is a cross-sectional view of the container portion shown in FIG. 2, taken along axis A-A of FIG. 2.

FIG. 4 of the drawing is a cross-sectional view of the container portion shown in FIG. 2, taken along axis B-B of FIG. 2.

FIG. 5 of the drawing shows the container of FIG. 2, positioned in the bowl or heating chamber of an herbal vaporizer.

### DETAILED DESCRIPTION OF THE INVENTION

Various non-limiting examples of embodiments of the present invention are described in detail in this section.

In a basic aspect, the invention comprises a thermally stable, chemically inert matrix material, the matrix material being highly porous or fibrous, with a great many interstitial channels or spaces and a very high surface area to volume ratio, in combination with a medicament.

The matrix material is generally adapted to receive, store and support the medicament on the surface of the matrix material and within at least some of the channels or interstitial spaces that are formed inside the matrix material.

The matrix material is also adapted to release the volatilizable active ingredients when the matrix material is heated to a temperature above the vaporization point of the active ingredients.

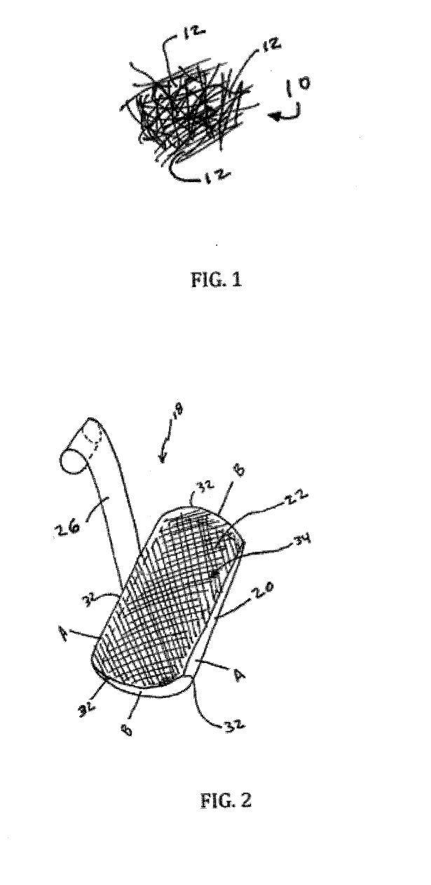

As shown in FIG. 1, the matrix material 10 may be in the form of a bunched mass of a highly fibrous material, and is preferably sintered or fused quartz wool. The matrix materially is thermally stable and chemically inert. Channels or interstitial spaces 12 are formed within the mass of matrix material. Fused quartz wool has properties that are similar to pure quartz glass, with fibers 14 that maintain their usable mechanical properties up to about 1050° C. Fused quartz wool generally consists of woven basic fibers that are non-flammable and incombustible and that have excellent torsion and bending resistance and excellent thermal conductivity.

Other materials could be used for a matrix material, provided they are (1) thermally stable, (2) chemically inert, and (3) have a great many voids, interstitial spaces or channels within the matrix, communicating with the surface of the mass of matrix media, such that the mass of material has a surface area-to volume ratio that is very high.

Because the matrix material is inflammable and non-combustible, after it has been used, it can be heated to high temperatures, for example, in an oven or kiln or with a hot torch, to drive off or combust any remaining medicament or other foreign substances that are still present in or on the matrix material. This forms a pristine, clean, sanitized matrix material that can then be re-loaded with another dose of a medicament, and re-used, again and again.

Some types of matrix materials, such as fibrous quartz wool, are quite fragile. Therefore, they may preferably be combined with a protective container for containing the matrix media. Like the matrix material, the protective container should be thermally stable and chemically inert. It must be permeable to the exiting vapors of the volatilized active ingredients. For example, a portion or all of the container's surface can comprise a gas permeable screen or mesh, or some other gas permeable material, to allow vapors to exit the container.

The surface of the container should also be permeable by various types of substances, to allow dosages of liquids, pastes, resins or waxes to be introduced into the matrix material through the permeable portion of the container. Particularly when the medicament is in the form of a resin, paste or wax, it might be necessary or advisable to heat the container and the medicament, to aid in the permeation of the medicament into the channels or interstitial spaces of the matrix media.

The container can have any of a variety of different shapes or sizes. It may contain multiple doses of the active ingredients in the medicament, but it will preferably contain only a single dose. A container for a single dose of medicament can generally be quite small. The container can and should be small enough to fit into the bowl or heating chamber of a pipe, water pipe or herbal vaporizer.

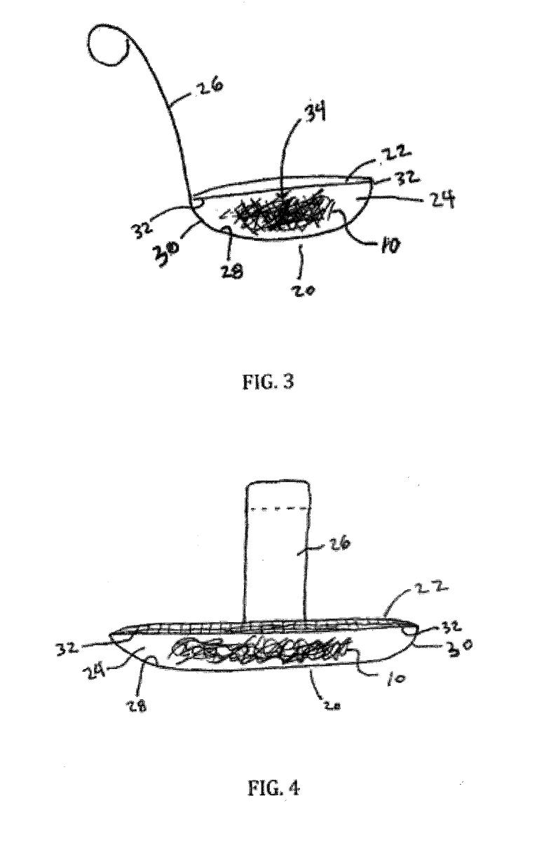

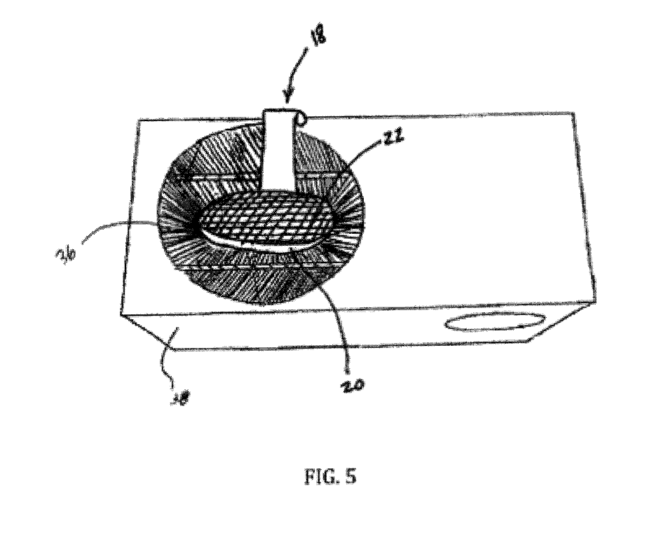

FIGS. 2-4 show an embodiment of an apparatus that comprises a matrix material 10 inside a container portion 18. As shown in FIGS. 2-4, the container portion 18 in the embodiment shown generally comprises a lower support platform 20 and an upper mesh portion 22. As shown in FIGS. 3 and 4, the lower support platform 20 and the mesh portion 22 define a closed interior volume 24 within the container 18. A stem or handle 26 can be attached to the outer surface of the container, for example, at the perimeter of the lower support platform 20.

The lower support platform 20 provides a generally flat, planer upper surface 28, on which to rest the matrix material 10, as discussed below. The lower support platform 20 can have any shape, consistent with the objects stated above, but preferably is shaped in the form of a generally rectangular pan or trough having thin, generally upwardly extending sidewalls 30, which may be curved, straight or faceted. The lower support platform 20 can be made using a flat plate material, such as stainless steel, or, alternatively, can be made using a Mesh material, such as stainless steel mesh, which is preferably similar to the upper mesh portion 22. The upper surfaces of the sidewalls 30 form a continuous edge 32, which defines an opening 34 between the adjacent edges. The opening 34 leads into the interior volume 24 of the container 18, which is defined by and between the support surface 28 and the sidewalls 30. The mesh portion 22 is attached over the opening 34, around the edge 32, and is fixed or welded all around the edge 32 using a spot welder or another suitable technique.

With the sealing of the mesh portion 22 onto the edge 32, the opening 34 is closed. This creates a closed interior volume 24 between the mesh 22 and the lower support platform 20. The matrix material 10 is contained inside the closed interior volume.

In a typical embodiment, the closed interior volume will be formed between the lower support platform 20, the mesh 22 and the upwardly extending sidewalls 30 of the lower support platform 20. In one preferred embodiment, generally shown in FIGS. 2-4, embodiment, the lower support platform is generally rectangular in shape, and is approximately 0.5 cm wide on the narrower side, 1 cm wide on the wider side, and with sidewalls 30 that are approximately 1-3 mm tall. This embodiment would be around the size of a dime and about as tall as two stacked dimes.

The lower support platform 20 can have any of a variety of different shapes that are capable of providing sufficient interior volume 24 when the mesh 22 is fastened onto the lower support platform 20.

It is important that all the components of the apparatus, including the lower support platform 20, the mesh 22 and the matrix material 10, be made using materials that are thermally stable and chemically inert. A preferred material for the lower support platform is stainless steel having a thickness of between about 0.0005 and about 0.012 inches, although any of a variety of different materials, such as other metals, metal alloys, ceramic or glass could also be used. A preferred material for the mesh portion is stainless steel, with a grid size between about 200 mesh and about 450 mesh, and preferably around 325 mesh (that is, 325 crossing wires per inch of length, with a wire diameter of about 0.0011 inches).

The mesh 22 is attached to the upper edges 32 of the sidewalls 30 of the lower support platform 20, to seal off the opening 34 that leads into the interior volume 24, to fully enclose the interior volume of the container with the matrix material inside the interior volume. The mesh 16 may be attached to the lower support portion 20 by welding with a spot welder (if both components are made of metal and can be welded) or by any other method that will withstand the types of temperatures to which the apparatus is exposed in use.

Alternatively, the mesh can be shaped to enable it to be snapped over the opening, around the perimeter of the edge 32, to enable the mesh to be removed and then snapped back into place over the sidewalls 30. Any of a variety of known approaches for fastening a removable upper cap onto a lower container could be used to fix the mesh 22 onto the lower support platform 20. For example, there could be a detent or outwardly extending ridge on some or all of the sidewalls 32, which mates with a corresponding groove or expanded portion on the outer perimeter of the mesh 22. These approaches would permit a user or manufacturer to remove the mesh 22 to replace the matrix material 10 and then to snap it back firmly and securely into place.

As shown in FIG. 5, the container portion of the apparatus is placed into the bowl 36 of a small, portable, hand-held herbal vaporizer, such as a product that is sold under the trademark LAUNCH BOX, by Magic-Flight, Inc. of San Diego, Calif. The container can also be place into the heating bowl or combustion chamber of a pipe or water pipe, where the container (and the matrix material with the medicament supported thereon) can be heated by a flame or other heat source to release the volatilizable components, which can then be inhaled through the vaporizer, pipe or water pipe in a normal fashion. Thus, the present apparatus takes advantage of a typical pipe, water pipe or evaporator to provide the heating of the matrix media and to provide a way for the user to inhale the released vapors with the active ingredients. For that reason, the container 18 is preferably sized to fit within the heating bowl or combustion chamber of a pipe or water pipe or evaporator, with a shape that preferably corresponds to the shape of the bowl or heating chamber, to help stabilize the container 18 within the bowl or heating chamber.

The disclosed apparatuses are generally useful in administering a variety of different types of medicaments, in a variety of different forms. Some of the medicaments include active ingredients that may be extracted from a wide variety of different herbs or other natural sources, including the following herbs, with the vaporization temperatures shown in Table 1 below:

TABLE 1

| | Vaporization |
|---|---|
| | Temperature |
| Medicament Source | (° F.) |
| Blue Lotus | 212-257 |
| Chamomile | 212-257 |
| Lavender | 212-257 |
| St. John's Wort | 212-302 |
| Damiana | 302-347 |
| Cannabis | 338-392 |
| Kava Kava | 347-392 |
| Maca Root | 302-392 |

The disclosed apparatuses can be used with a variety of different medicaments. The use of the apparatuses is described below in connection with the administration of kava. Kava, or kava kava, is a medicament that is extracted from the roots of the kava kava plant, Piper methysticum, a crop of the western Pacific. The active ingredients, called kavalacones, are reputed to have a mild sedative and anti-anxiety effect. Extracted Kava can be chewed, put into beverages or dried and put into pills.

With respect to the present invention, the active ingredients of Kava, the kavalactones, are volatilizable. Thus, they can be extracted and introduced into a matrix media, using a liquid, a viscous resin or some other form or in some other material that is a suitable carrier. A typical dose will vary between about 5 mg and about 150 mg, depending on a variety of factors including the concentration of the active ingredients in the medicament and the size of the person to whom it is being administered. This enabling disclosure is not intended to constitute any medical recommendation for any medicament or any dosage of any medicament.

If the matrix media is being used alone, without any container, the medicament can be metered onto the matrix media using any dispenser that is capable of administering small, accurate amounts of materials, such as a metered syringe. The matrix media can then be placed into the bowl or heating chamber of a pipe, water pipe or herbal vaporizer, the kavalactones can be vaporized by heating them, and the user can inhale the vaporized kavalactones through the pipe, water pipe or vaporizer apparatus.

If a fragile matrix media is being used in combination with a container, for example, as shown in FIGS. 4-7, the carrier (e.g., liquid, resin, wax or paste) with the kavalactones is metered onto the surface of the mesh 28, and permeates through the mesh 28 and into the matrix media 10. If the carrier is too viscous to permeate freely through the mesh and into the matrix media, the apparatus can be warmed or heated (for example, with an electric heater), to a temperature substantially below the vaporization point listed above. This will generally reduce the viscosity of the medicament and improve its permeation through the screen and into the matrix media.

Once the medicament has permeated through the mesh and into the matrix media, the entire apparatus can be stored for an extended period. Refrigeration of the units might be necessary or appropriate, depending on the nature of the medicament.

Preferably, each unit is preloaded with a single dose of the medicament, particularly if the matrix media is inside a container, as shown in FIGS. 2-5. These pre-loaded doses can be stored for an extended period, and are small and durable enough to be carried in a user's purse, car glove compartment, backpack or the like, along with a hand-held pipe or vaporizer for dispensing the dose.

After a pre-loaded container has been used, it can be cleaned by heating the unit, for example, with the flame of a torch, to a temperature that is sufficiently high to combust or degrade any foreign substances that remain on the apparatus, and to drive any volatile components from the apparatus, including from the matrix media.

After the unit has been cleaned, another dose of a medicament can be loaded into the apparatus, by placing the medicament onto the permeable mesh and allowing it to permeate through the mesh and into the matrix media, as described above. The cycle (load, use, clean, re-load, use, clean) can be repeated through multiple cycles, and potentially for years depending on the apparatus's durability.

What is claimed is:

1. An apparatus for selectively releasing a volatilizable active ingredient in a medicament, the apparatus comprising:
(a) a mass of matrix material, the matrix material being thermally stable and chemically inert and having a high plurality of void spaces, interstitial spaces or channels therein, the mass of matrix material being adapted to receive the medicament with the medicament being supported on the surface of portions of the matrix media and permeating into at least some of the void spaces, interstitial spaces or channels; and
(b) a container having an interior volume for containing the mass of matrix material, the container being made of materials that are thermally stable and chemically inert, with at least a portion of the container being gas permeable so as to enable vapors of the active ingredients to exit the container through the gas permeable portion upon heating of the matrix material to a temperature above the vaporization temperature of the active ingredients.

2. The apparatus of claim 2, wherein the matrix material comprises fused quartz wool.

3. The apparatus of claim 2, wherein the container comprises a lower support platform and a gas permeable upper mesh portion, the support platform having sidewalls that extend generally upward from the support platform to an upper edge, with the support platform and the sidewalls defining at least a portion of the interior volume of the container and the upper edges of the sidewalls defining an opening into the interior volume; and wherein the permeable mesh portion extends over the opening and is fastened to the sidewalls to close off the interior volume with the matrix material inside the interior volume.

4. The apparatus of claim 3, wherein the container is shaped and sized to fit into the heating bowl of a pipe, water pipe or herbal vaporizer.

5. The apparatus of claim 4, wherein the container is generally flat and rectangular in shape.

6. The apparatus of claim 4, the container further comprising an upwardly extending handle portion that can be grasped when placing the container into the heating chamber or lifting it from the heating chamber.
